(19) United States
(12) Patent Application Publication
(10) Pub. No.: US 2017/0319500 A1
(43) Pub. Date: Nov. 9, 2017

(54) SELF-ASSEMBLED GELS FOR CONTROLLED DELIVERY OF BIOLOGICS AND LABILE AGENTS

(71) Applicant: The Brigham and Women's Hospital, Inc., Boston, MA (US)

(72) Inventors: Jeffrey Karp, Brookline, MA (US); Nitin Joshi, Quincy, MA (US); Xueyin He, Ontario (CA); Julian Amirault, Andover, MA (US); Brittany Laramee, Shrewsbury, MA (US); Kai Slaughter, Ontario (CA)

(21) Appl. No.: 15/589,895

(22) Filed: May 8, 2017

Related U.S. Application Data
(60) Provisional application No. 62/332,673, filed on May 6, 2016.
(60) Provisional application No. 62/332,643, filed on May 6, 2016.

(51) Int. Cl. A61K 9/48; A61K 9/70; A61K 9/00; A61M 31/00; A61K 9/50

(52) U.S. Cl. CPC A61K 9/4833; A61K 9/70; A61K 9/0019; A61K 9/5015; A61M 31/002

(57) ABSTRACT

Gels are formed based on generally recognized as safe (GRAS) low molecular weight amphiphilic molecules in a self-assembly process with limited or no heating. A selective range of ratios between an organic solvent and water, or an aqueous solution, in the medium, allows for the GRAS low molecular weight amphiphiles to form a homogeneous self-supporting gel encapsulating agents to be delivered under very mild conditions. Proteins including enzymes, antibodies, and serum albumin are loaded in the self-assembled gels to provide sustained and/or responsive delivery. The encapsulated proteins retain at least 70%, 80%, or 90% of their activity over days in various storage conditions.

### CROSS-REFERENCE TO RELATED APPLICATIONS

This application claims benefit of and priority to U.S. Provisional Application Nos. 62/332, 643 and 62/332,673, filed on May 6, 2016, which are hereby incorporated herein by reference in their entirety.

### STATEMENT REGARDING FEDERALLY SPONSORED RESEARCH OR DEVELOPMENT

This invention was made with government support under Grant No. W81XWH-14-1-0229 awarded by the Department of Defense. The government has certain rights in the invention.”

### FIELD OF THE INVENTION

The disclosed technology is generally in the field of controlled delivery of drug, and more particularly, relates to responsive delivery from self-assembled gels that do not compromise the activity of biologics and labile agents.

### BACKGROUND OF THE INVENTION

Use of self-assembling gels which are stable in vivo for drug delivery are described in US2017/0000888. Self-assembly in forming molecularly defined, high-ordered structures largely relies on non-covalent interactions. Structures formed from self-assembly are capable of entrapping molecules in solution during the assembly process, resulting in injectable carriers suitable for delivery of hydrophobic and hydrophilic agents. One common approach to forming self-assembled gel is with amphiphilic compounds which in theory may spontaneous assemble due to hydrophilic-hydrophobic interactions.

Heating is generally necessary to homogeneously disperse these amphiphilic agents in a medium, such that upon cooling ordered nano and micro structures are assembled and, macroscopically, a self-supporting gel is formed. The gel is useful as a vehicle for drug delivery, as a reservoir for controlled release of drug agents, and may possess desirable biochemical and mechanical properties as scaffold for tissue repair.

However many protein therapeutics are heat-sensitive. Many nucleic acids, small compounds, peptide, and other biologically derived components are also sensitive or labile to heat. Insuring a high loading amount of biologically active agent in these self-assembled gels is challenging.

Therefore, it is an object of the present invention to provide a self-assembled gel composition and a process for making with limited to no heating.

It is another object of the present invention to provide a self-assembled gel composition that maintains the activity of encapsulated agents for controlled delivery.

### SUMMARY OF THE INVENTION

Gels are formed based on generally recognized as safe (GRAS) low molecular weight amphiphilic molecules (termed “gelators”) in a self-assembly process with limited to no heating. Biologic agents as well as heat-sensitive agents can be loaded in the self-assembled gels to provide sustained and responsive delivery. A combination of an organic solvent and water or an aqueous solution (termed “gelation medium”), at a selective ratio, is effective to dissolve gelators and active agents into a homogeneous solution. The organic solvent and water (or an aqueous solution) may be added simultaneously, sequentially, or pre-mixed before addition to the gelators. In a first embodiment as demonstrated in Example 1, heating is not required to homogeneously dissolve gelators in either the organic solvent or the gelation medium, the organic solvent and water may be added simultaneously, sequentially, or pre-mixed. Biologic agents are incorporated in the gelation medium without being exposed to any denaturing temperature. In a second embodiment as demonstrated in Example 2, where heating facilitates dissolution of gelators in an organic solvent, the organic solution with the gelators as the solutes (termed “gelator solution”) does not solidify when cooled to about body temperature (37° C.) or room temperature (25° C.). Water or an aqueous solution suspending biologic agents is subsequently added to the cooled gelator solution to initiate gelation.

Formed gel contains a high loading of biologic agents without exposing these agents to the denaturing temperature. Formed gel is self-supporting, i.e., stable to inversion. Encapsulated biologic agents or other therapeutic, prophylactic, or diagnostic agents maintain at least 70%, 80%, or 90% of their activity or intrinsic structural configurations in the self-gel for at least 1 day, 2 days, 3 days, 1 week, 2 weeks, 1 month, or greater in refrigeration, ambient temperature, or at 37° C. Generally increasing the concentration of gelators increases the encapsulation efficiency of a therapeutic, prophylactic, or diagnostic agent in the self-assembled gel.

The organic solvent is generally water-miscible, such that a homogeneous solution with water or with an aqueous solution can be formed. Exemplary organic solvents for gelation include dimethyl sulfoxide (DMSO), dipropylene glycol, propylene glycol, hexyl butyrate, glycerol, acetone, dimethylformamide, tetrahydrofuran, dioxane, acetonitrile, ethanol, and methanol. A preferred class of organic solvents are alcohols, especially fatty alcohols.

The GRAS low molecular weight amphiphilic molecules are generally at least 3, 4, 5, or 6 wt/vol %. The organic solvent is generally at least 15%, 16%, 17%, 18%, 19%, 20%, 21%, 22%, 23%, 24%, or 25% in volume of the gelation medium. Depending on the types of gelators and the organic solvent in the gelation medium, a minimal volume percentage of the organic solvent is required to permit gelation. Too little organic solvent may result in no gelation (i.e., flowable mass or precipitates of the gelators) or solidification/hardening of the gelators, preventing gelation from happening once water or an aqueous solution is added. Too much organic solvent may also prevent gelation from occurring, or damage labile biological agents to be encapsulated. Typically, the organic solvent is no greater than 50%, 60%, 70%, or 75% in volume of the gelation medium.

The GRAS low molecular weight amphiphilic molecules can have degradable linkage such that in vivo environment or other stimuli may trigger the release of encapsulated therapeutic agents from the gel. In the absence of these stimuli (e.g., enzyme, pH, temperature), the gel does not exhibit burst release and has minimal leakage, so that encapsulated agent is released over a prolonged period of time.

The organic solvent in the self-assembled gel can be removed or substantially removed to a level where the residual amount is within the stated limit of pharmaceutical products by the U.S. Food and Drug Administration (FDA). Drying, solvent exchange, or lyophilization may be used to remove excessive organic solvent and/or excel or unencapsulated agents.

The self-assembled gel may be suspended in a pharmaceutically acceptable carrier for administration. It may also be homogenized, sonicated, or otherwise dispersed as particles, which may be dried, suspended, or administered in gel. The self-assembled gel, its suspension formulation, or particle formulation may also be incorporated into a bandage, wound dressing, patch, or in a syringe or catheter.

The self-assembled gel, its suspension formulation, or particle formulation, is administered to deliver an effective dosage of a therapeutic, prophylactic, or diagnostic agent to alleviate, prevent or treat one or more symptoms of a disease or disorder.

### BRIEF DESCRIPTION OF THE DRAWINGS

FIG. 1 is a bar graph showing the activity (%) of lysozyme after encapsulation in ascorbyl palmitate for different amounts of time (hours) in different storage temperatures.

FIG. 2 is a bar graph showing the encapsulation efficiency (%) of fluorescein isothiocyanate-labeled bovine serum albumin (FITC-BSA) in ascorbyl palmitate particles or fibers formed with 30% DMSO or 50% DMSO in water.

FIG. 3 is a bar graph showing the encapsulation efficiency (%) of FITC-labeled immunoglobulin G (IgG-FITC) in ascorbyl palmitate particles formed with 30% DMSO or 50% DMSO.

FIG. 4 is a line graph showing the cumulative release (%) of BSA-FITC from ascorbyl palmitate gels that were incubated in phosphate buffered saline (PBS) or in PBS with esterase.

FIG. 5 is a line graph showing the cumulative release (%) of encapsulated bovine serum albumin (labeled with fluorescein isothiocyanate; BSA-FITC) over time (days) from ascorbyl palmitate gel suspended in phosphate buffered saline in a dialysis bag into a sink medium containing a large amount of phosphate buffered saline.

FIG. 6 is a line graph showing the cumulative release (%) of encapsulated FITC-labeled immunoglobulin G (IgG-FITC) over time (days) from ascorbyl palmitate gel suspended in phosphate buffered saline in a dialysis bag into a sink medium containing a large amount of phosphate buffered saline.

FIG. 7 is a bar graph showing the encapsulation efficiency (%) of a small interfering RNA (siRNA) over the concentrations (wt/vol %) of ascorbyl palmitate in a gelation medium.

### DETAILED DESCRIPTION OF THE INVENTION

### I. Definitions

The term “gelators” refer to molecules that can self-assemble through non-covalent interactions, such as hydrogen-bonding, van der Waals interactions, hydrophobic interactions, ionic interactions, pi-pi stacking, or combinations thereof, in one or more solvents. The gelators can form a gel by rigidifying the solvent through, for example, capillary forces. Gelators can include hydrogelators (e.g., gelators that form hydrogels) and organo-gelators (e.g., gelators that form organo-gels). In some embodiments, gelators can form both hydrogels and organo-gels.

The term “self-assembling” refers to the capability of molecules to spontaneous assemble, or organize, to form a higher ordered structure such as hydrogel or organo-gel in a suitable environment.

The term “hydrogel” refers to three-dimensional (3-D) networks of molecules covalently (e.g., polymeric hydrogels) or non-covalently (e.g., self-assembled hydrogels) held together where water is the major component. Gels can be formed via self-assembly of gelators or via chemical crosslinking of gelators. Water-based gelators can be used to form hydrogels. Organo-gelators are gelators that form gels (organogels) in solvents where organic solvents are the major component.

The term “organo-gel” refers to 3-D networks of molecules covalently (e.g., polymeric hydrogels) or non-covalently (e.g., self-assembled hydrogels) held together where an organic solvent is the major component. Gels can be formed via self-assembly of gelators or via chemical crosslinking of gelators.

The term “co-assembly”, refers to the process of spontaneous assembly, or organization of at least two different types of molecules to form a high ordered structure such as hydrogel or organo-gel in a suitable environment, where molecules in the structure are generally organized in an ordered manner

The term “organic solvent” refers to any carbon-containing substance that, in its liquid phase, is capable of dissolving a solid substance. Exemplary organic solvents commonly used in organic chemistry include toluene, tetrahydrofuran, acetone, dichloromethane, and hexane.

The term “water-miscible” refers to an solvent that mixes with water, in all proportions, to form a single homogenous liquid phase. This includes solvents like dimethyl sulfoxide (DMSO), tetrahydrofuran, acetone, ethanol, methanol, and dioxane, but generally excludes solvents such as hexane, oils, and ether. It also excludes solvents that have some, very limited miscibility or solubility in water such as ethyl acetate and dichloromethane, which are practically considered immiscible.

The term “percent (%) encapsulated” or “encapsulation percentage” is generally calculated as % encapsulated=weight of encapsulated drug÷weight of total of initial drug (encapsulated +unencapsulated)×100%.

The term “encapsulation efficiency (EE)”is generally calculated as EE (%)=experimental/measured drug loading÷theoretical drug loading×100%.

The term “pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

The terms “biocompatible” and “biologically compatible,” as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

The term “molecular weight,” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

The term “hydrophobic,” as used herein, refers to the property of lacking affinity for or repelling water. For example, the more hydrophobic a polymer (or polymer segment), the more that polymer (or polymer segment) tends to not dissolve in, not mix with, or not be wetted by water.

The term “surfactant” as used herein refers to an agent that lowers the surface tension of a liquid.

The term “therapeutic agent” refers to an agent that can be administered to prevent or treat one or more symptoms of a disease or disorder. Therapeutic agents can be nucleic acids or analogs thereof, a small molecule (mw less than 2000 Daltons, more typically less than 1000 Daltons), peptidomimetic, protein, or peptide, carbohydrate or sugar, lipid, or a combination thereof. In some embodiments, cells or cellular materials may be used as therapeutic agents.

The term “treating” or “preventing” a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The term “therapeutically effective amount” refers to an amount of the therapeutic agent that, when incorporated into and/or onto the self-assembled gel composition, produces some desired effect at a reasonable benefit/risk ratio applicable to any treatment. The effective amount may vary depending on such factors as the disease or condition being treated, the particular formulation being administered, the size of the subject, or the severity of the disease or condition.

The terms “incorporated” and “encapsulated” refers to incorporating, formulating, or otherwise including an agent into and/or onto a composition, regardless of the manner by which the agent or other material is incorporated.

### II. Composition

1. Gelators

Formation and use of self-assembling gels which are stable in vivo for drug delivery are described in US2017/0000888. GRAS amphiphilic gelators suitable for self-assembly to form gel are generally less than 2,500 Da, and may preferably be enzyme-cleavable. The GRAS amphiphile gelators can self-assemble into gels based micro-/nano-structures (e.g., lamellar, micellar, vesicular, or fibrous structures).

In some embodiments, the GRAS amphiphile gelators are ascorbyl alkanoate, sorbitan alkanoate, triglycerol monoalkanoate, sucrose alkanoate, glycocholic acid, or any combination thereof.

The alkanoate can include a hydrophobic C₁-C₂₂ alkyl (e.g., acetyl, ethyl, propyl, butyl, pentyl, caprylyl, capryl, lauryl, myristyl, palmityl, stearyl, arachidyl, or behenyl) bonded via a labile linkage (e.g., an ester, a carbamate, a thioester and an amide linkage) to an ascorbyl, sorbitan, triglycerol, or sucrose molecule. For example, the ascorbyl alkanoate can include ascorbyl palmitate, ascorbyl decanoate, ascorbyl laurate, ascorbyl caprylate, ascorbyl myristate, ascorbyl oleate, or any combination thereof. The sorbitan alkanoate can include sorbitan monostearate, sorbitan decanoate, sorbitan laurate, sorbitan caprylate, sorbitan myristate, sorbitan oleate, or any combination thereof. The triglycerol monoalkanoate can include triglycerol monopalmitate, triglycerol monodecanoate, triglycerol monolaurate, triglycerol monocaprylate, triglycerol monomyristate, triglycerol monostearate, triglycerol monooleate, or any combination thereof. The sucrose alkanoate can include sucrose palmitate, sucrose decanoate, sucrose laurate, sucrose caprylate, sucrose myristate, sucrose oleate, or any combination thereof.

In some embodiments, the GRAS amphiphile gelators include ascorbyl palmitate, sorbitan monostearate, triglycerol monopalmitate, sucrose palmitate, or glycocholic acid.

Representative low molecular weight GRAS amphiphile gelators include vitamin precursors such as ascorbyl palmitate (vitamin C precursor), retinyl acetate (vitamin A precursor), and alpha-tocopherol acetate (vitamin E precursor).

In some forms, a GRAS amphiphile gelator is formed by synthetically conjugating one or more saturated or unsaturated hydrocarbon chains having C₁ to C₃₀ groups with a low molecular weight, generally hydrophilic compound, through esterification or a carbamate, anhydride, and/or amide linkage. The range C₁ to C₃₀ includes C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉ etc. up to C₃₀ as wells as ranges falling within C₁ to C₃₀, for example, C₁ to C₂₉, C₂ to C₃₀, C₃ to C₂₈, etc.

In some embodiments, alpha tocopherol acetate, retinyl acetate, retinyl palmitate, or a combination thereof, can co-assemble with the gelators.

Typically to form a viscous gel stable to inversion (e.g., resist flow when inverted), greater than 3%, 4%, 5% (wt/vol) or more gelators are included in a liquid medium. The gels can include, independently, from 0.01 (e.g., from 0.05, from 0.5, from one, from two, from three, from five, from 10, or from 15) to 40 percent (to 40, to 30, to 20, to 15, to 10, to five, to three, to two, to one, to 0.5, to 0.05) of GRAS amphiphile gelators by weight per volume.

In some forms, the self-assembled gel compositions include an enzyme-cleavable, generally recognized as safe (GRAS) first gelator having a molecular weight of 2500 or less and a non-independent second gelator that is also a GRAS agent. Non-independent gelators do not form self-supporting gel at the concentration that would typically form self-supporting gel if combined with an enzyme-cleavable GRAS gelator. Exemplary non-independent second gelators include alpha tocopherol acetate, retinyl acetate, and retinyl palmitate. The non-independent gelators co-assemble with the GRAS first gelators to form the self-assembled gels.

The gels can include, independently, from about three to a maximum of 30-40 percent, more preferably about 4% to 10% by weight gelator per volume of gel. Above 30-40% the gel will begin to precipitate out of solution or become less injectable.

2. Gelation medium

The liquid medium for the gelators to form self-assembled gel generally includes a two-solvent system of an organic solvent and water (or an aqueous salt solution), or an aqueous-organic mixture solvent system.

In a first embodiment, a GRAS gelator and a therapeutic, prophylactic, or diagnostic agent are mixed and/or dissolved to homogeneity in a co-solvent medium including both water (or an aqueous buffer or salt solution) and a water-miscible organic solvent, to form a gelation solution.

In a second embodiment, a GRAS gelator is dissolved initially in an organic solvent to form a solution with the GRAS gelator as the solute (termed “gelator solution”). A therapeutic or prophylactic agent, for example, biologics, is dissolved in the gelator solution or in an aqueous solution such as pure water or an aqueous buffer or salt solution (depending on the hydrophobicity or hydrophilicity of the agent). An aqueous solution or the aqueous solution containing the therapeutic or prophylactic agent is then mixed (e.g., quickly via pipetting, stirring, or vortexing) with the gelator solution to form a gelation solution.

Preferably no heating is needed, or, if necessary, heating to about body temperature (37° C.) generates a homogeneous self-supporting gel that is stable to inversion. In other embodiments, the gelation solution is heated to complete dissolution, followed by cooling to about 37° C. or room temperature around 20° C.-25° C. The gel should not be heated above 37° C. or room temperature, to avoid loss of activity of the encapsulated agent.

In the first embodiment, gelation takes place upon the formation of a gelation solution without heating. In the second embodiment, gelation takes place as the heated gelation solution is cooled. Leaving the gel on a stable surface for about one to two hours at room temperature results in a consistent self-supporting gel. Self-supporting gel comprises orderly assembled micro-or nano-structures with minimal precipitates. This is generally confirmed using optical or electron microscopy.

The organic solvent is selected based on the solubility of gelators therein, its polarity, hydrophobicity, water-miscibility, and in some cases the acidity. Suitable organic solvents include water-miscible solvent, or solvent that has an appreciable water solubility (e.g., greater than 5 g/100 g water), e.g., DMSO, dipropylene glycol, propylene glycol, hexyl butyrate, glycerol, acetone, dimethylformamide (DMF),tetrahydrofuran, dioxane, acetonitrile, alcohol such as ethanol, methanol or isopropyl alcohol, as well as low molecular weight polyethylene glycol (e.g., 1 kD PEG which melts at 37° C.). In other forms, the self-assembled gel compositions can include a polar or non-polar solvent, such as water, benzene, toluene, carbon tetrachloride, acetonitrile, glycerol, 1,4-dioxane, dimethyl sulfoxide, ethylene glycol, methanol, chloroform, hexane, acetone, N, N′-dimethyl formamide, ethanol, isopropyl alcohol, butyl alcohol, pentyl alcohol, tetrahydrofuran, xylene, mesitylene, and/or any combination thereof.

Preferred organic solvents for gelation include dimethyl sulfoxide (DMSO), dipropylene glycol, propylene glycol, hexyl butyrate, glycerol, acetone, dimethylformamide, tetrahydrofuran, dioxane, acetonitrile, ethanol, and methanol. Another class of organic solvents, fatty alcohols or long-chain alcohols, are usually high-molecular-weight, straight-chain primary alcohols, but can also range from as few as 4-6 carbons to as many as 22-26, derived from natural fats and oils. Some commercially important fatty alcohols are lauryl, stearyl, and oleyl alcohols. Some are unsaturated and some are branched.

Generally, the amount of an organic solvent is no more than 1:1, 1:2, 1:3,1:4, 1:5, 1:6, 1:7, 1:8, 1:9, 1:10, or less in volume compared to the volume of an aqueous solution (e.g., water, aqueous buffer, aqueous salt solution, optionally containing a therapeutic agent). That is, the volume amount of an organic solvent in the total amount of liquid as used in forming a homogenous gel with high drug loading is generally less than about 50%, 33%, 25%, 20%, 17%, 14%, 12.5%, 11%, 10%, or 9%, and significantly less, typically less than 1%, for particles.

Gelators and organic solvents are selected at an appropriate gelator concentration and appropriate volume and ratio of the aqueous-organic mixture solvent system, or both, to form self-supporting gel. The gelator solution should not solidify or precipitate at 37° C. before the addition of an aqueous solution containing biologics or other therapeutic agent. Increasing the amount of the organic solvent or reducing the concentration of gelators in the organic solvent may prevent solidification of the gelator solution. When the gelator solution (in an organic solvent) is mixed with the aqueous solution containing biologics or other therapeutic agent, a self-supporting gel stable to inversion is formed, (following heating if necessary), rather than flowable mass/aggregates.

Following formation of self-supporting gels, the organic solvent in the gel may be removed to a residual level suitable for pharmaceutical applications. One or more purification techniques such as dialysis, centrifugation, filtration, drying, solvent exchange, or lyophilization, can be used. Residual organic solvent is within the stated limit of pharmaceutical products by the U.S. Food and Drug Administration (FDA) or below the acceptance criteria by U.S. Pharmacopeia Convention, International Conference on Harmonization guidance. For example, dicloromethane is below 600 ppm, methanol below 3,000 ppm, chloroform below 60 ppm; and within the limit by GMP or other quality based requirements.

3. Therapeutic, Prophylactic and Diagnostic Active Agents

The gel compositions are suitable for delivery of one or more therapeutic, prophylactic or diagnostic agents to an individual or subject in need thereof. Therapeutic, prophylactic and diagnostic agents may be proteins, peptides, sugars or polysaccharides, lipids or lipoproteins or lipopolysaccharids, nucleic acids (DNA, RNA, siRNA, miRNA, tRNA, piRNA, etc.) or analogs thereof, or small molecules (typically 2,000 D or less, more typically 1,000 D or less, organic, inorganic, natural or synthetic) to repair or regenerate cartilage or treat disorders therewith.

In some forms, gelators may be prodrugs that hydrolytically or enzymatically degrade and release active agents.

In other forms, a therapeutic, prophylactic, or diagnostic agent may be physically entrapped, encapsulated, or non-covalently associated with the nanostructures in the gel composition. The therapeutic, prophylactic, or diagnostic agents may be covalently modified with one or more gelators, one or more stabilizers, or be used as a gelator. Alternatively, they are incorporated into the assembled ordered lamellar, vesicular, and/or nanofibrous structures of the gel composition or positioned on the surface of the assembled structures.

Suitable actives include immunomodulatory molecules such as steroids, non-anti-inflammatory agents, chemotherapeutics, anesthetics, analgesics, anti-pyretic agents, anti-infectious agents such as antibacterial, antiviral and antifungal agents; chemotherapeutics, vitamins, therapeutic RNAs such as small interfering RNA, microRNA, PiRNA, ribozymes, and nucleotides encoding proteins or peptides, and in some cases, cells.

Exemplary proteins to encapsulate in self-assembled gel include enzymes (e.g., lysozyme), antibodies (e.g., immunoglobulin, monoclonal antibody, and antigen binding fragments thereof), growth factors (e.g., recombinant human growth factors), antigens, and peptides such as insulin.

In some embodiments, the self-assembled gel include genome editing nucleic acids that encode an element or elements that induce a single or a double strand break in the target cell's genome, and optionally a polynucleotide. An exemplary strand break inducing element is CRISPR/Cas-mediated genome editing composition. CRISPR is an acronym for Clustered Regularly Interspaced Short Palindromic Repeats; and they are often associated with genes which code for proteins that perform various functions related to CRISPRS, termed CRISPR-associated (“Cas”) genes. A typical CRISPR/Cas system allows endogenous CRISPR spacers to recognize and silence exogenous genetic elements, either as a prokaryotic immune system or adopted as a genome editing tool in eukaryotes. (see, for example, Cong, Science, 15:339(6121):819-823 (2013) and Jinek, et al., Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423.

In the context of an endogenous CRISPR system, formation of a CRISPR complex (including a guide sequence of CRISPR hybridized to a target sequence and complexed with one or more Cas proteins) results in cleavage of one or both strands in or near the target sequence. In the context of introducing exogenous CRISPR system into a target cell, one or more vectors may be included in the self-assembled gels to drive expression of one or more elements of a CRISPR system such that they form a CRISPR complex at one or more target sites in the target cell. The vectors may include one or more insertion sites (e.g., restriction endonuclease recognition sequence), a regulatory element operably linked to an enzyme-coding sequence encoding a CRISPR enzyme such as a Cas protein, or one or more nuclear localization sequences. Alternatively, a vector encodes a CRISPR enzyme that is mutated with respect to a corresponding wild-type enzyme such that the mutated CRISPR enzyme lacks the ability to cleave one or both strands of a target polynucleotide containing a target sequence.

Resources are available to help practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequence. For example, a practitioner interested in using CRISPR technology to target a DNA sequence (identified using one of the many available online tools) can insert a short DNA fragment containing the target sequence into a guide RNA expression plasmid. Detection of accumulation in the nucleus may be performed by any suitable technique, such as fusion to the CRISPR enzyme a detectable marker, immunohistochemistry to identify protein, or enzyme activity assay.

In one embodiment, two or more agents are encapsulated or loaded in the self-assembled gel. One agent may potentiate the efficacy of another encapsulated agent.

In another embodiment, the self-assembled gel compositions include a mixture of therapeutic agents (e.g., a cocktail of proteins) for continuous delivery to a tissue or a cell in need thereof.

Diagnostic agents which can be included in the self-assembled gel composition include paramagnetic molecules, fluorescent compounds, magnetic molecules, and radionuclides. Suitable diagnostic agents include, but are not limited to, x-ray imaging agents and contrast media. Radionuclides can be used as imaging agents. Examples of other suitable contrast agents include gases or gas emitting compounds, which are radiopaque.

The agent is generally encapsulated at a concentration between about 1 mg/mL and about 200 mg/mL in the self-assembled gel.

4. Optional stabilizing agent

In some embodiments, agents enhancing blood stability and/or reducing the rate of disassembly of nanostructures after administration are included in the composition. Stabilizing agents typically impart rigidity, increase the packing density, and/or enhance the strength of assembled structures, thus altering the phase transition process and transitioning temperature, and/or modulating the surface properties of assembled particles to reduce or prevent protein adhesion or accumulation.

Additional materials can be included with the therapeutic agents to modify release and bioactivity, such as polyalcohols poly(ethylene oxide) and poly(ethylene glycol), copolymers and acrylated derivatives thereof, celluloses such as carboxy methylcellulose, and combinations thereof.

Generally, the stabilizing agents diminish the rate of reduction in the size of the assembled particles or nanoparticles when placed in a serum solution, whereas compositions without stabilizing agents substantially decrease the hydrodynamic size in serum solutions in about 30 minutes. Stabilizing agents allow for more than 50%, 60%, 70%, 80%, 90%, 95%, 99% of the assembled nanostructures to have less than 1%, 5%, 10%, 15%, 20%, or 30% reduction in the hydrodynamic sizes in at least one, two, three, four, 12, 24, or 48 hours in incubation with serum at 37° C.

In general, the molecules that can rigidify the self-assembled lamellae will usually be hydrophobic molecules, molecules that can change surface properties, like small chain hydrophilic polymers, and/or molecules that can modify the surface charge (charged molecules).

In some embodiments, the stabilizing agents are co-assembled with gelators in the formation of assembled gel compositions. These stabilizing agents are generally incorporated into the lamellar, micellar, vesicular, and/or fibrous structures by encapsulation, integrated, entrapment, insertion or intercalation. Generally, inclusion of 10-30 mole % of co-assembly type, stabilizing agents allows for the assembled nanoparticles to maintain about 80% or more of the original size when incubated over a period of two to four hours in serum solutions.

Blood proteins including albumin can interact with irregularities in the assembled lamellar, micellar, vesicular, and/or fibrous structures, such as those that exist at the phase boundaries, resulting in a higher rate of disassembly of particles or the higher structured nanoparticles or bulk hydrogel. Other exemplary stabilizing agents include sterols, phospholipids, and low molecular weight therapeutic compounds that are typically hydrophobic. Suitable sterols include cholesterol, corticosteriods such as dihydrocholesterol, lanosterol, β-sitosterol, campesterol, stigmasterol, brassicasterol, ergocasterol, Vitamin D, phytosterols, sitosterol, aldosterone, androsterone, testosterone, estrogen, ergocalciferol, ergosterol, estradiol-17alpha, estradiol-17beta, cholic acid, corticosterone, estriol, lanosterol, lithocholic acid, progesterone, cholecalciferol, cortisol, cortisone, cortisone acetate, cortisol acetate, deoxycorticosterone and estrone and fucosterol. Suitable phospholipids include dipalmitoyl phosphatidyl choline and distearoyl phosphatidyl choline. The phospholipids typically co-assemble with one or more gelators in forming the ordered lamellar and/or fibrous structures. Other stabilizing agents include, but are not limited to, lysophospholipids (including lyso PC, 2-hexadecoxy-oxido-phosphoryl)oxyethyl-trimethyl-azanium), gangliosides, including GM1 and GT1b, sulfatide, sphingophospholipids, synthetic glycopholipids such as sialo-lactosyl, phospholipids, including DOPE, DOPS, POPE, DPPE, DSPE, lipophilic drugs such as cytosine arabinoside diphosphate diacyglycerol, proteins such as cytochrome b5, human high density lipoprotein (HDL), human glycophorin A, short chain hydrophilic polymers, including polyethylene glycol (PEG) and their derivatives with lipids, bile acids include taurocholic acid, desoxycholic acid, and geicocholic acid, 1,1′-dioctadecyl 3,3,3′,3′-tetramethyl-indocarbocyanine percholorate (DiI), DiR, DiD, fluorescein isothiocynate, tetramethylrhodamine isothiocyanate, rhodamine B octadecyl ester perchlorate and N′-Octadecylfuorescein-5-thiourea. Sterols generally co-assemble with one or more gelators, inserting into the ordered lamellar, micellar, vesicular, and/or fibrous structures. Sterols by themselves are not gelators and cannot form gel compositions on their own.

Suitable low molecular weight therapeutic, prophylactic and/or diagnostic agents used as stabilizing agents for the gel compositions are generally hydrophobic, of a low molecular weight (e.g., less than 2,500 Da), such as docetaxel and steroids and other hydrophobic a gents such as dexamethasone, or a combination of agents.

In other embodiments, the stabilizing agents are encapsulated in the assembled composition, typically throughout the gel composition, rather than insertion or intercalation into the lamellar, micellar, vesicular, and/or fibrous structures. Generally, inclusion of between 5 and 15 mole % stabilizing agents allows for the assembled nanostructures to maintain about 80% or more of the original size when incubated over a period of two to four hours in serum solutions.

In some embodiments, therapeutic, prophylactic and/or diagnostic agents may diminish the size of the assembled nanostructures when placed in a blood or serum solution, where more than 50%, 60%, 70%, 80%, 90%, 95%, 99% of the nanostructures in incubation with serum at 37° C. have less than 1%, 5%, 10%, 15%, 20%, or 30% reduction in the hydrodynamic sizes in at least one, two, three, four, 12, 24, or 48 hours, compared to gel composition without the active agents. An exemplary hydrophobic, chemotherapeutic agent, docetaxel, may stabilize the nanostructures formed from gelators when encapsulated at a molar percentage of 2%, 4%, 6%, 8%, and 10%, and all values in the range, between the active agent and the gelators.

5. Properties

### Mechanical Property & Injectability

With self-assembled gel compositions, no gravitational flow is observed upon inversion of a container at room temperature for at least 10 seconds, and in some cases, for about 1 hour, 3 hours, 1 day, 2 days, 3 days, one week or longer. A self-assembled gel is homogeneous and stable to inversion, unlike heterogeneous materials that is a mixture of gelled regions (non-flowable) and non-gelled, liquid regions (flowable). A self-assembled gel is also different from liposome or micelle suspensions. Liposome or micelles suspensions are not self-supporting and can flow when the container is inverted.

In some embodiments, the self-assembled gel compositions have recoverable rheological properties, i.e., self-assembled gel is shear-thinning, suitable for injection, and recovers to a self-supporting state after cessation of a shear force. The self-supporting state generally features an elastic modulus of from 10 to 10,000 Pascal and greater than a viscous modulus. Due to non-covalent interactions for the assembly of gelators and cationic agents, a bulk gel may deform and be extruded under a shear force (e.g., during injection), and the gelators and cationic agents re-assemble upon cessation of shear forces to a self-supporting, stable-to-inversion state (e.g., elastic modulus G′ greater than viscous modulus G″).

Alternatively, the self-assembled gel composition is injectable as suspended in a pharmaceutically acceptable carrier, i.e., a suspension medium, being a fibrous suspension state.

Another form of the self-assembled gel is a microparticle or nanoparticle, where the bulk self-supporting gel is homogenized, sonicated, or otherwise dispersed in a suspension medium and further collected.

### Micro- and/or Nano-Structures

The agents can be encapsulated within or between the nanostructures, can be non-covalently bonded to the nanostructures, or both.

The hydrophobic parts and the hydrophilic parts of the gelator molecules can interact to form nanostructures (lamellae, sheets, fibers, particles) of gelator molecules. The therapeutic agent inserts and forms part of the nanostructures, is encapsulated in the gel, or both. In some embodiments, when the gels are hydrogels, the hydrophobic portions of gelators are located in the inner regions of a given nanostructures, and hydrophilic portions are located at the outer surfaces of the nanostructure. In some embodiments, when the gels are organogels, the hydrophobic portions of gelators are located in the outer regions of a given nanostructure, and hydrophilic portions are located at the inner surfaces of the nanostructure. The nanostructure can have a width of from about three (e.g., from about four) to about five (e.g., to about four) nanometers and a length of several microns (e.g., one micron, two microns, three microns, four microns, five microns, ten microns, twenty microns, or twenty five microns) or more. Several tens or hundreds of lamellae can bundle together to form nanostructures, such as fibers and sheet-like structures.

In some embodiments, the nanostructures include nanoparticles, micelles, liposome vesicles, fibers, and/or sheets. In some embodiments, The nanostructures can have a minimum dimension (e.g., a thickness, a width, or a diameter) of 2 nm or more (e.g., 50 nm or more, 100 nm or more, 150 nm or more, 200 nm or more, 250 nm or more, 300 nm or more, 350 nm or more) and/or 400 nm or less (e.g., 350 nm or less, 300 nm or less, 250 nm or less, 200 nm or less, 150 nm or less, 100 nm or less, or 500 nm or less). In some embodiments, the nanostructures (e.g., fibers, sheets) have a length and/or width of several microns (e.g., one micron, two microns, three microns, four microns, five microns, ten microns, twenty microns, or twenty five microns) or more. The nanostructures can aggregate into networks, and/or be in the form of a liquid crystal, emulsion, fibrillar structure, or tape-like morphologies. When the nanostructures are in the form of fibers, the fibers can have a diameter of about 2 nm or more, and can have lengths of hundreds of nanometers or more. In some embodiments, the fibers can have lengths of several microns (e.g., one micron, two microns, three microns, four microns, five microns, ten microns, twenty microns, or twenty five microns) or more.

### Degradation (Cleavable Linkage)

Stimuli evoking release can be present due to the characteristics at the site of administration or where release is desired, for example, tumors or areas of infection. These may be conditions present in the blood or serum, or conditions present inside or outside the cells, tissue or organ. These are characterized by low pH and the presence of degradative enzymes. The gel compositions may be designed to disassemble only under conditions present in a disease state of a cell, tissue or organ, e.g., inflammation, thus allowing for release of an agent at targeted tissue and/or organ. This is an alternative or may be used in combination to gel erosion-mediated and passive diffusion-mediated release of agent.

This responsive release is based on linkages formed from degradable chemical bonds (or functional groups) and/or tunable non-covalent association forces (e.g., electrostatic forces, van der Waals, or hydrogen bonding forces). In some embodiments, these linkages are (1) degradable covalent linkage between the hydrophilic segment and the hydrophobic segment of an amphiphile gelator, (2) positioned in a prodrug-type gelator, which upon cleavage releases an active drug, and/or (3) covalent linkage or non-covalent association forces between a gelator and a therapeutic agent. The cleavage or dissociation of these linkages result in (1) more rapid or greater release of the encapsulated or entrapped agents compared to passive diffusion-mediated release of agent; and/or (2) converts prodrug gelator into active drug for release.

Stimuli evoking release includes intrinsic environment in vivo and user-applied stimulation, for example, enzymes, pH, oxidation, temperature, irradiation, ultrasound, metal ions, electrical stimuli, or electromagnetic stimuli. A typical responsive linkage is cleavable through enzyme and/or hydrolysis, based on a chemical bond involving an ester, an amide, an anhydride, a thioester, and/or a carbamate. In some embodiments, phosphate-based linkages can be cleaved by phosphatases or esterase. In some embodiments, labile linkages are redox cleavable and are cleaved upon reduction or oxidation (e.g., —S—S—). In some embodiments, degradable linkages are susceptible to temperature, for example cleavable at high temperature, e.g., cleavable in the temperature range of 37-100° C., 40-100° C., 45-100° C., 50-100° C., 60-100° C., 70-100° C. In some embodiments, degradable linkages can be cleaved at physiological temperatures (e.g., from 36 to 40° C., about 36° C., about 37° C., about 38° C., about 39° C., about 40° C.). For example, linkages can be cleaved by an increase in temperature. This can allow use of lower dosages, because agents are only released at the required site. Another benefit is lowering of toxicity to other organs and tissues. In certain embodiments, stimuli can be ultrasound, temperature, pH, metal ions, light, electrical stimuli, electromagnetic stimuli, and combinations thereof.

### Release

The gel compositions can be designed for controlled degradation at a site or after a period of time, based on the conditions at the site of administration. Compared to free agent in a solution, the encapsulated agent releases from the self-assembled gel much slower, for example, less than 30% of encapsulated agent is released in the first three days and less than 70% in seven days. In the presence of a stimulus such as an enzyme, self-assembled gel formed from a gelator with an enzyme-degradable linkage releases the agent more rapidly, compared to the gel in a medium lacking the enzyme.

6. Formulations

The self-assembled gel composition with affinity to connective tissues may be prepared in dry powder formulations or liquid formulations.

Generally the formulation is sterilized or sterile. For example, a sterile formulation can be prepared by first performing sterile filtration of gelators, cationic agents, as well as agents to be encapsulated, followed by processes of making in an aseptic environment. Alternatively, all processing steps can be performed under non-sterile conditions, and then terminal sterilization (e.g., gamma or E-beam irradiation) can be applied to the formed particles or lyophilized product.

Dry formulations contain lyophilized self-assembled gel compositions where solvent is removed, resulting in xerogels. Xerogels can be in a powder form, which can be useful for maintaining sterility and activity of agents during storage and for processing into desired forms. As xerogels are solvent free, they can have improved shelf-life and can be relatively easily transported and stored. To lyophilize self-assembled gels, the gels can be frozen (e.g., at −80° C.) and vacuum-dried over a period of time to provide xerogels.

Alternatively, a dry formulation contains dry powder components of gelators, cationic agents, one or more therapeutic agents, which are stored in separate containers, or mixed at specific ratios and stored. In some embodiments, suitable aqueous and organic solvents are included in additional containers. In some embodiments, dry powder components, one or more solvents, and instructions on procedures to mix and prepare assembled nanostructures are included in a kit.

Liquid formulations contain self-assembled gel composition suspended in a liquid pharmaceutical carrier. In some forms, self-assembled gel is suspended or resuspended in aqueous media for ease of administration and/or reaching a desired concentration for minimizing toxicity.

Suitable liquid carriers include, but are not limited to, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human. The liquid formulations may be isotonic relative to body fluids and of approximately the same pH, ranging from about pH 4.0 to about pH 8.0, more preferably from about pH 6.0 to pH 7.6. The liquid pharmaceutical carrier can include one or more physiologically compatible buffers, such as a phosphate or bicarbonate buffers. One skilled in the art can readily determine a suitable saline content and pH for an aqueous solution that is suitable for an intended route of administration.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propylp-hydroxybenzoate.

### III. Method of Making

1. Making Self-Supporting Gel for Delivery of Biologics and Labile Agents.

Generally, a water-miscible organic solvent dissolves gelators to form a gelator solution and optionally a therapeutic, prophylactic, or diagnostic agent. An aqueous medium (e.g., water, hypotonic solution, isotonic solution, or hypertonic solution) optionally containing a therapeutic, prophylactic, or diagnostic agent is added and quickly mixed with the gelation solution. At appropriate volume ratios of the organic solvent and the aqueous solution, gelation begins as soon as the aqueous medium is mixed with the gelator solution. Over time, the gel becomes consistent. Gelation is deemed complete when the gel is self-supporting and stable to inversion at room temperature for at least 10 seconds, and in some cases, for about 10 minutes, 30 minutes, 1 day, 3 days, 1 week, 2 weeks, 3 weeks, or longer, i.e., not “runny” or flow due to gravity, no precipitates, and no aggregates. A self-assembled gel is homogeneous and stable to inversion, unlike heterogeneous materials that are a mix of gelled regions (non-flowable) and non-gelled, liquid regions (flowable).

Alternatively, a gelator and a therapeutic, prophylactic, or diagnostic agent are mixed and/or dissolved to homogeneity in a co-solvent medium including both water (or an aqueous buffer or salt solution) and a water-miscible organic solvent, to form a gelation solution.

Generally no heating is required for the formation of homogeneous gel, thus preserving the activity of biologics and other heat-sensitive agents. In some embodiments, moderate heating to body temperature (about 37° C.) improves homogeneity of gelators and agents in the medium. In other embodiments, heating is needed to dissolve gelators and therapeutic agents in the mixture-solvent medium, followed by cooling for gelation to take place.

2. Purification

Distillation, filtration, dialysis, centrifugation, other solvent exchange techniques, vacuum, drying, or lyophilization may be used in one or more repeated processes to remove unencapsulated excess agent and organic solvent from the gels to below the stated limit of pharmaceutical product requirements.

Generally a purification medium is one suitable for administration, such that the solvent of the gel is at least partially replaced with the purification medium.

Generally, a process to make the self-assembled gel composition includes combining gelators, cationic agents, therapeutic agents, and solvents to form a mixture; heating or sonicating the mixture; stirring or shaking the mixture for a time sufficient to form a homogeneous solution; and cooling the homogenous solution for a time sufficient to enable the formation of self-assembled gel compositions.

3. Suspension into fibrous mixture and processing into particles

The self-assembled gels in some embodiments are suspended in a pharmaceutically acceptable for ease of administration to a patient (e.g., by drinking or injection) and/or to provide a desired drug concentration to control toxicity.

In some forms, the bulk gel is suspended in water, phosphate buffered saline, or other physiological saline, which is homogenized or sonicated to break up the bulk gel into particles which retain the fibrous nanostructures formed in the bulk gel. These particles may be collected, stored, and reconstituted prior to use in a suitable medium and at an appropriate concentration for administration. Different types of gel particles loaded with different amounts or types of therapeutic agents may be combined.

In some embodiments, particles are nanoparticles having a hydrodynamic diameter between 100 nm and 990 nm, preferably between 500 nm and 900 nm, and the nanoparticles maintain at least 50, 60, 70 or 80% of the size in serum over a period of at least two hours.

In other embodiments, particles are microparticles having a diameter ranging from 1 μm to a couple hundred millimeters.

4. Sterilization

A sterile formulation is prepared by first performing sterile filtration of the process solutions (e.g., drug and gelator solutions), followed by gel preparation, suspension, purification and lyophilization under aseptic procession conditions. Alternatively, all processing steps can be performed under non-sterile conditions, and then terminal sterilization (e.g., gamma or E-beam irradiation) can be applied to the lyophilized hydrogel product. Sterile solution for resuspension can also be prepared using similar methods.

### IV. Methods of Using

The gel composition, the fibrous suspension, or the gel particle suspension, optionally encapsulating biologics or other therapeutic, prophylactic, or diagnostic agents, can be administered through various known regional delivery techniques, including injection, implantation, inhalation using aerosols, and topical application to the mucosa, such as the oral or buccal surfaces, nasal or pulmonary tracts, intestinal tracts (orally or rectally), vagina, or skin. In situ self-assembly of stabilized nanostructures allows for regional delivery of the compositions and stimuli-responsive delivery of active agents, especially to areas of infection, trauma, inflammation or cancer.

Delivered biologics or other agents can be controllably released from the gel compositions in response to stimuli for targeted release. In the absence of stimuli, the agent is released in a sustained manner with little to no burst release. For example, encapsulated agents can be gradually released over a period of time (e.g., hours, one day, two days, three days, a week, a month, or more). Depending on the parameters, release can be delayed or extended from minutes to days to months, for example, when gel compositions are administered under physiological conditions (a pH of about 7.4 and a temperature of about 37° C.).

For example, parenteral administration includes administration to a patient intravenously, intradermally, intraperitoneally, intrapleurally, intratracheally, intramuscularly, subcutaneously, subjunctivally, by injection, and by infusion.

The compositions are useful for improving targeting efficiency, efficacy, safety, and compliance benefiting from single dose, prolonged action or tissue-specific formulations, compared to therapeutics delivered in its free solution form. In some embodiments, the compositions can be useful to release therapeutic agents that correlate with different stages of tissue regeneration.

Exemplary diseases or disorders to be treated with the stabilized assembled nanostructures include, but are not limited to, allergy (e.g. contact dermatitis), arthritis, asthma, cancer, cardiovascular disease, diabetic ulcers, eczema, infections, inflammation, mucositis, periodontal disease, psoriasis, respiratory pathway diseases (e.g., tuberculosis), vascular occlusion, pain, graft versus host diseases, canker sores, mucositis, bacterial conditions, viral conditions.

In some forms, the self-assembled gel composition is used in a method of preventing or treating one or more symptoms any one of the exemplary diseases or disorders in a subject by administering an effective amount of the self-assembled gel composition to deliver an effective amount of therapeutic, prophylactic, or diagnostic agents.

The present invention will be further understood by reference to the following non-limiting examples.

### Examples

### Example 1

### Without Heating, a Threshold Amount of DMSO is Required for Ascorbyl Palmitate to Form Homogeneous Gel.

Methods

Ascorbyl palmitate was prepared with a total volume of 200 μL including a first organic solvent, DMSO, and a second solvent, ultrapure water, at 10 w/v %. The DMSO was at a volume percentage of 20%, 25%, 30%, 50% of the combined volume including DMSO and ultrapure water.

Results

The vials containing the samples were inverted for visual examination to determine if homogeneously gelled.

Ascorbyl palmitate in the solvent mixture with 20% DMSO formed precipitates, i.e., heterogeneous materials that was a mix of gelled regions (non-flowable) and non-gelled, liquid regions (flowable with some precipitates in there).

Ascorbyl palmitate in the solvent mixture with 25% DMSO, ascorbyl palmitate in the solvent mixture with 30% DMSO, and ascorbyl palmitate in the solvent mixture with 50% DMSO, all formed gel that did not flow when the vial was inverted. These gels were macroscopically homogeneous, as no non-gelled liquid phase was observed.

Optical microscopy analysis showed ascorbyl palmitate in the solvent mixture with 30% DMSO was the optimal combination out of the four groups, as indicated by nanofibrous structure with minimal aggregation. Using too much DMSO will still self-assemble AP upon addition of water; however, the gel may not be self-supporting, i.e. it would be more like a free flowing suspension.

### Example 2

### Selective Amphiphiles Form Gels in a Two-Solvent Medium.

Methods

A GRAS amphiphile (ascorbyl palmitate, triglycerol monostearate TG18, sucrose stearate, sucrose palmitate, tetradecyl maltoside, or sorbitan monostearate) was added to the vial: for a final concentration of 10 w/v % or 6 w/v % in a total amount of 200 μL it liquid media including an organic solvent and ultrapure water.

60 μl of DMSO, dipropylene glycol, or propylene glycol was added to the vial. The vial was heated until dissolution of amphiphile; for amphiphiles that dissolved without heating, the heating step was omitted. The vial was allowed to cool to 37° C. in a 37° C. incubator; for amphiphiles that dissolved without heating, the cooling step could be omitted.

140 μl of ultrapure water without or with biologics was added, and the contents in the vial were immediately stirred to mix. The vial was later undisturbed on a flat surface for 1-2 hours.

Therefore the first solvent (DMSO, dipropylene glycol, or propylene glycol) was 30% (v/v) of the total liquid volume.

Results

### (1) A Two-Solvent Medium of DMSO and Water:

At a final 10 w/v % in DMSO-water, ascorbyl palmitate (AP), triglycerol monostearate (TG18), sucrose stearate (SS), and sucrose palmitate (SP) formed self-supporting gel (did not flow when vial was inverted) with DMSO-water as the solvent, but tetradecyl maltoside (TDM) and sorbitan monostearate (SMS) did not (Table 1). Sucrose palmitate took a longer time (overnight) for gelation than AP, TG18, and SS. 10 w/v % TDM solubilized in DMSO-water as flowable liquid. 10 w/v % SMS precipitated with DMSO-water, and did not form a self-supporting gel.

TABLE 1
Amphiphiles (at 10 w/v %) with 30% DMSO in a DMSO-water system.

| ascorbyl | triglycerol | sucrose | sucrose | tetradecyl | sorbitan |
|---|---|---|---|---|---|
| palmitate | monostearate | stearate | palmitate | maltoside | monostearate |
| (AP) | (TG18) | (SS) | (SP) | (TDM) | (SMS) |
| Gelled | Gelled | Gelled | Gelled | No | No gelation |
| | | | | gelation | |

Next, the minimal amount of DMSO in a DMSO-water system for a gelator to form gel was determined (Table 2). TG18 and SS was prepared separately at a final concentrating of 10% (w/v) in a DMSO-water system of a total liquid volume of 200 μL, where the amount of DMSO varied between 15% and 30% (v/v).

In a DMSO-water system with 10 w/v % TG18, DMSO was required at more than 15% (v/v) of the total solvent volume to allow the formation of self-supporting gel. 10% (w/v) TG18 in 30% (v/v) DMSO formed self-supporting gel; 10% (w/v) TG18 in 20% (v/v) DMSO formed self-supporting gel; and 10% (w/v) TG18 in 15% (v/v) DMSO did not form self-supporting gel. Optical microscopy of self-supporting gels showed ordered structures with no precipitates.

In a DMSO-water system with 10 w/v % SS, DMSO was required at more than 20% (v/v) of the total solvent volume to allow the formation of self-supporting gel. 10% (w/v) SS in 30% (v/v) DMSO formed self-supporting gel. Optical microscopy of self-supporting gel prepared from 10% (w/v) SS in 30% (v/v) DMSO showed ordered structures with no precipitates.

However, for a final 10% (w/v) SS in a total liquid volume containing 20 v/v % DMSO and 80 v/v % ultrapure water, SS in DMSO solidified at 37° C. before the addition of water, therefore no gel could be formed. That is, 20 mg sucrose stearate in 40 μL DMSO solidified at 37° C. during cooling after the mixture was heated, prior to the addition of 160 μL water, and therefore no gel could be formed.

AP-DMSO is the only combination that does not require heating.

TABLE 2
Summary of minimum DMSO amounts for gelation of different
GRAS amphiphiles at a final concentration of 10 w/v %.

| | | | 15% v/v |
|---|---|---|---|
| | 30% v/v DMSO | 20% v/v DMSO | DMSO |
| TG18 | Gelled | Gelled | No gelation |
| SS | Gelled | Solidified before addition of | |
| | | water; no gel was formed. | |

With a given volume percentage of an organic solvent, gelation also depended on the amount of a specific gelator, i.e., the initial concentration of gelator when it was first dissolved in the organic solvent.

Solidification did not happen when sucrose stearate was first dissolved in DMSO to prepare for a final 6% (w/v) SS with the addition of water, when DMSO was 20 v/v % in the DMSO-water system: 12 mg sucrose stearate in 40 μL DMSO did not solidify at 37° C., and the addition of 160 μL led to gelation eventually. This was unlike the previous case where in preparation for a final amount of 10% (w/v) SS in an overall 20 v/v % DMSO and 80 v/v % ultrapure water, sucrose stearate solidified at 37° C. in DMSO before the addition of water,

### (2) A Two-Solvent Medium of Dipropylene Glycol (DPG) and Water:

At a final concentration of 6 w/v % in a dipropylene glycol-water system, ascorbyl palmitate (AP) and triglycerol monostearate (TG18) formed self-supporting gel (did not flow when vial was inverted), but sucrose palmitate (SP) did not form self-supporting gel (Table 3). 6% SP precipitated in dipropylene glycol, which flowed when the vial was inverted.

TABLE 3
Amphiphiles (at 6 w/v %) with 30% dipropylene glycol in a
dipropylene glycol-water system prepared with heating.]

| Ascorbyl palmitate | triglycerol | |
|---|---|---|
| (AP) | monostearate (TG18) | sucrose palmitate (SP) |
| Gelled | Gelled | No gelation |

Next, the minimal amount of DPG in a DPG-water system for a gelator to form gel was determined (Table 4). Ascorbyl palmitate (AP) and triglycerol monostearate (TG18) hydrogel at a final concentration of 6% w/v was prepared with a total solvent volume of 200 4, where the amount of DPG varied between 15% and 30% (v/v).

In a DPG-water system with an overall 6 w/v % ascorbyl palmitate (AP), DPG was required at more than 15% (v/v) of the total solvent volume to allow the formation of self-supporting gel. 6% w/v AP in 30% v/v DPG formed self-supporting gel; 6% w/v AP in 20% v/v DPG formed self-supporting gel; but 6% w/v AP in 15% v/v DPG did not form self-supporting gel. Optical microscopy of self-supporting gels showed ordered structures with no precipitates.

In a DPG-water system with an overall 6 w/v % TG18, DPG was required at more than 20% (v/v) of the total solvent volume to allow the formation of self-supporting gel. 6% (w/v) TG18 in 30% DPG (v/v) formed self-supporting gel. Optical microscopy of 6% (w/v) TG18 in 30% (v/v) DPG gels showed ordered structures with no precipitates.

However, for a final 6% (w/v) TG18 in a total liquid volume containing 20 v/v % DPG and 80 v/v % ultrapure water, TG18 solidified in DPG at 37° C. before the addition of water, therefore no gel could be formed. That is, 12 mg TG18 in 40 μL DPG solidified at 37° C., prior to the addition of 160 4 water, and therefore no gel could be formed.

In this example, all required heating. Solidified refers to the solid mass that is obtained after cooling the amphiphile solution in solvent 1. Solidified mass just contains amphiphile molecules dispersed homogeneously throughout the solvent, and is not a self-assembled structure. Solidification is undesirable during gelation process.

Gel is a self-assembled structure that is formed after solvent 2 is added to amphiphile solution in solvent 1.

TABLE 4
Summary of minimum DPG amounts for gelation of different
GRAS amphiphiles at 6 w/v %.

| | 30% v/v DPG | 20% v/v DPG | 15% v/v DPG |
|---|---|---|---|
| Ascorbyl | Gelled | Gelled | Did not form |
| palmitate | | | self-supporting |
| (AP) | | | gel |
| TG18 | Gelled | Solidified before addition | |
| | | of water; no gel was | |
| | | formed. | |

### (3) A Two-Solvent Medium of Propylene Glycol (PG) and Water:

At a final concentration of 6 w/v % in 200 μL liquid containing 30% propylene glycol and 70% water, ascorbyl palmitate (AP) formed self-supporting gel (did not flow when vial was inverted); triglycerol monostearate (TG18) formed self-supporting mass but with granular aggregates; but sucrose stearate (SS) and sucrose palmitate (SP) did not form self-supporting gel (Table 5).

TABLE 5
Amphiphiles (at 6 w/v %) with 30% (v/v) propylene glycol in a
propylene glycol-water system.

| Ascorbyl | Triglycerol | Sucrose | Sucrose |
|---|---|---|---|
| palmitate | monostearate | stearate | palmitate |
| Gelled | No; containing | No gel | No gel |
| | granular aggregates | | |

Next, the minimal amount of propylene glycol (PG) in a PG-water system for a gelator to form gel was determined (Table 6). Ascorbyl palmitate (AP) at an overall concentration of 6% w/v was prepared with a total solvent volume of 200 μL, where the amount of PG varied between 15% and 30% (v/v). Ultrapure water was the other liquid medium.

In a PG-water system with overall 6 w/v % ascorbyl palmitate (AP), 30%, 20%, and 15% v/v PG all formed self-supporting gel; whereas 10% v/v PG did not support the formation of self-supporting gel. Optical microscopy of self-supporting gels showed ordered structures with no precipitates.

TABLE 6
Summary of PG amounts for gelation of ascorbyl palmitate at 6 w/v %.

| 30 v/v % PG | 20 v/v % PG | 15 v/v % PG | 10 v/v % PG |
|---|---|---|---|
| Gelled | Gelled | Gelled | No gel |

### Example 3

### Lysozymes or Amylase Encapsulated in Ascorbyl Palmitate Gels With DMSO-Water as the Medium Retained a High Encapsulation Efficiency and Activity Over Days.

Methods

50 mg ascorbyl palmitate (AP) was dissolved in 150 μL DMSO and heated. AP solution in DMSO was allowed to cool down to 37° C. 350 μL of 5 mg/mL lysozyme or amylase stock in water was added to the AP solution and mixed to make an overall 3.5 mg/mL lysozyme or amylase-loaded gel.

After gel was formed, fibers were produced by adding 2 ml water. The suspension was centrifuged at 10,000 rpm for 10 min and the pellet was resuspended in water to get lysozyme loaded particles. Encapsulation efficiency was determined using HPLC and activity of the enzyme in supernatant was determined using lysozyme or amylase activity kit.

Results

Lysozyme was encapsulated at an efficiency of 79.3%. The activity of lysozyme after encapsulation was 89%, as determined immediately after gel preparation.

Amylase was encapsulated at an efficiency of 70.5%. The activity of amylase retained at 92% after encapsulation, as determined immediately after gel preparation.

The gel preparation process, as well as the suspension and particle-making processes, was detrimental to the activity of encapsulated enzymes, or proteins.

Lysozyme-loaded gels were also stored at different temperatures (25° C., 37° C., and 4° C.) immediately following gel preparation (t=0), and the activity of lysozyme was determined at different time points (t=2, 4, 8, 24, 48, and 72 hours).

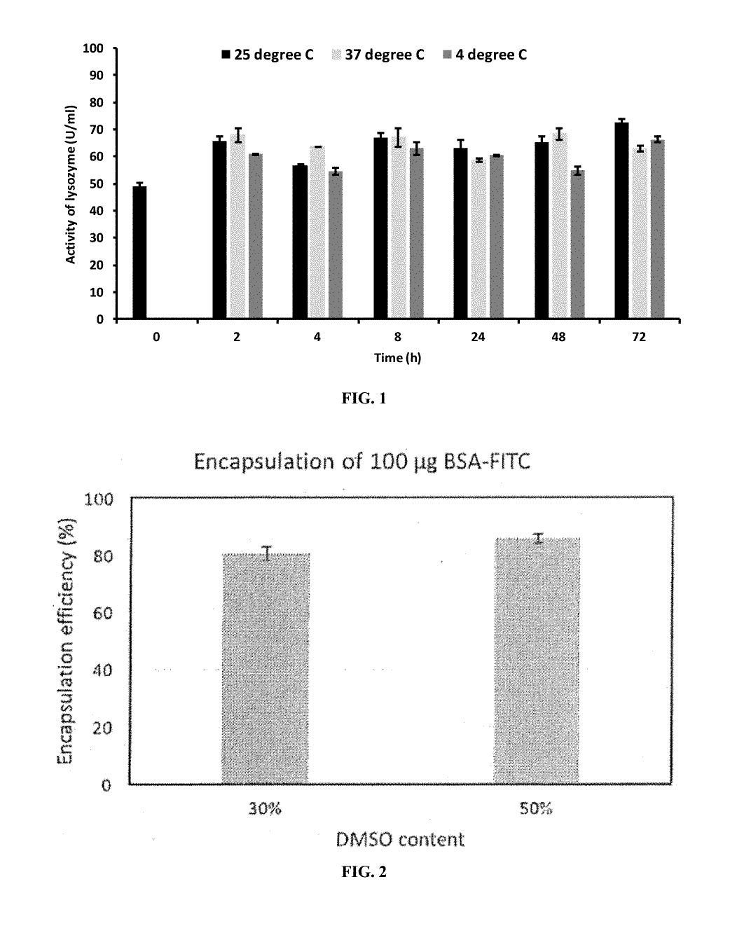

FIG. 1 shows the activity of lysozyme was maintained in the gel for at least 72 hours in all storage conditions.

### Example 4

### Gels Encapsulate Large Amounts of Protein for Enzyme-Responsive Release.

Methods

Bovine serum albumin (BSA) and immunoglobulin were labeled with fluorescein isothiocyanate (FITC) for ease of quantification, i.e. BSA-FITC and IgG-FITC. Gels were formed with a DMSO content of 30% or 50% without heating in a DMSO-water system as described above. Encapsulation efficiency is in reference to the fibers or particles derived from the gel, i.e. after centrifugation to remove the untrapped agent.

Results

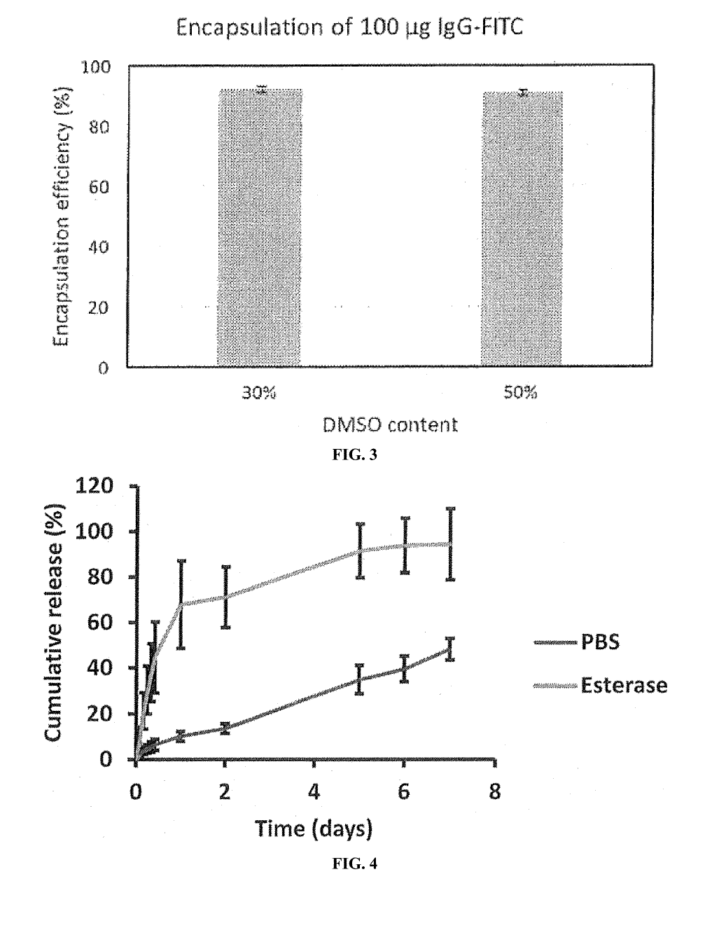

FIGS. 2 and 3 show ascorbyl palmitate gels encapsulated large amounts of bovine serum albumin (BSA) and antibodies (IgG), respectively.

DMSO content variation (30% and 50%) did not have a significant effect on encapsulation efficiency.

FIG. 4 shows FITC-labeled BSA was stably encapsulated in ascorbyl palmitate gel under a normal physiological condition and was released in response to an enzyme, esterase.

### Example 5

### Sustained Release of Proteins From Ascorbyl Palmitate Gels Prepared in A DMSO-Water System.

Methods

BSA-FITC loaded ascorbyl palmitate gels were prepared with different BSA-FITC concentrations (2.5 and 5 mg/ml).

BSA-FITC loaded hydrogel (200 μL) was placed in dialysis tubing (300 kD molecular weight cut-off, Spectrum Labs) and suspended in PBS (800 μL). The dialysis bags filled with hydrogel in the release medium were placed in a 20 mL sink medium (PBS), and incubated at 37° C. with a shaking speed of 150 rpm. At each time point, an aliquot (1 mL) of the sink medium was removed and replenished with the same volume of fresh PBS to ensure constant sink conditions. Aliquots were analyzed for fluorescence using a fluorescence plate reader.

IgG-FITC loaded hydrogel (200 μL) containing 0.5 mg/ml IgG-FITC was placed in dialysis tubing (300 kD molecular weight cut-off, Spectrum Labs) and suspended in PBS (800 μL). The dialysis bags filled with hydrogel in release medium were placed in 20 mL sink medium (PBS), and incubated at 37° C. with a shaking speed of 150 rpm. At each time point, an aliquot (1 ml) of the sink medium was removed and replenished with the same volume of fresh PBS to ensure constant sink conditions. Aliquots were analyzed for fluorescence using a fluorescence plate reader. Release of free IgG-FITC from dialysis bags was performed as a control

Results

FIG. 5 shows the slow sustained release of BSA-FITC from the gel suspension over 7 days.

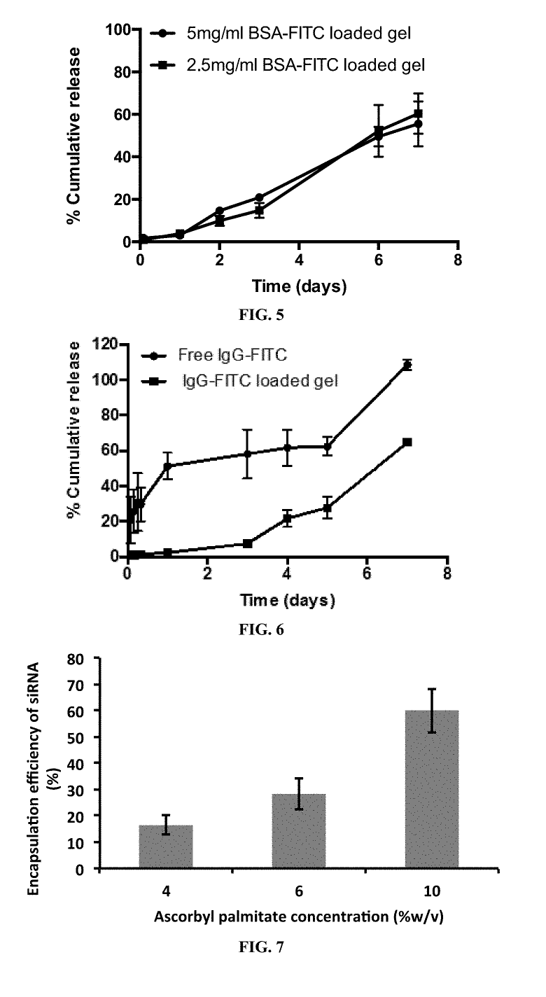

FIG. 6 shows the slow sustained release of labeled protein from the gel suspension over 7 days, as compared to burst and quick release of the free protein in its solution.

### Example 6

### Encapsulation of siRNA in Ascorbyl Palmitate Gels Prepared in a Propylene Glycol-Water System.

Methods

Ascorbyl palmitate (AP) was dissolved in 60 μL propylene glycol by heating. AP solution in propylene glycol was allowed to cool down to 37° C. 5 μL of CY®-3 (a cyanine dye) labelled GAPDH siRNA stock (50 μM) was diluted to 140 μL using RNAse free water and added to the AP solution with vigorous mixing using a pipette tip to form gel. GAPDH is the abbreviation for glyceraldehyde 3-phosphate dehydrogenase.

500 μL water was added to the gel followed by centrifugation. The pellet was dissolved in methanol, and the amount of CY®-3 labelled siRNA was quantified using a fluorescence plate reader to determine encapsulation efficiency. Encapsulation efficiency was determined in different hydrogels with varying concentration of AP (4% w/v, 6% w/v and 10% w/v).

Results

FIG. 7 shows the encapsulation efficiency of siRNA increased as the concentration of the gelator increased.

We claim:

1. A self-assembled gel composition for delivery of one or more therapeutic, prophylactic or diagnostic agents which lose activity when exposed to heating to above 37° C., comprising
generally recognized as safe (GRAS) gelators having a molecular weight of 2,500 or less, forming a hydrogel or organogel when heated then cooled in a solution comprising aqueous gelation medium and organic solvent, the gel comprising nanostructures, wherein the gel is stable for at least ten minutes to inversion at 25° C., and
therapeutic, prophylactic, or diagnostic agent incorporated within the gel and/or nanostructures therein, wherein the encapsulated agent has at least 50%, more preferably 80%, of the activity prior to encapsulation.

2. The gel composition of claim 1 formed from a homogeneous solution of gelator in the absence of heating to above 37° C.

3. The gel composition of claim 2 formed by heating the homogeneous solution to 37° C. or 25° C., then cooling.

4. The gel composition of claim 1, wherein the therapeutic, prophylactic, or diagnostic agent maintains at least 80% of its activity for at least three days at 4° C. or at body temperature (37° C.).

5. The gel composition of claim 1, wherein the organic solvent is greater than 10% in volume of the gelation medium.

6. The gel composition of claim 5, wherein the organic solvent comprises a solvent selected from the group consisting of dimethyl sulfoxide (DMSO), dipropylene glycol, propylene glycol, hexyl butyrate, glycerol, acetone, dimethylformamide, tetrahydrofuran, dioxane, acetonitrile, ethanol, and methanol.

7. The gel composition of claim 1, wherein the GRAS gelator is present in a concentration of at least 4 wt/vol % or greater in the gelation medium, and the organic solvent is between 15% and 50% in volume of the gelation medium.

8. The gel composition of claim 1, wherein the GRAS gelator is an ascorbyl alkanoate selected from the group consisting of ascorbyl palmitate, ascorbyl decanoate ascorbyl laurate, ascorbyl caprylate, ascorbyl myristate, ascorbyl oleate, and combinations thereof.

9. The gel composition of claim 1, wherein the GRAS gelator is triglycerol monoalkanoate selected from the group consisting of triglycerol monopalmitate, triglycerol monodecanoate, triglycerol monolaurate, triglycerol monocaprylate, triglycerol monomyristate, triglycerol monostearate, triglycerol monooleate, and combinations thereof.

10. The gel composition of claim 1, wherein the GRAS gelator is a sucrose alkanoate selected from the group consisting of sucrose palmitate, sucrose stearate, sucrose decanoate, sucrose laurate, sucrose caprylate, sucrose myristate, sucrose oleate, and combinations thereof.

11. The gel composition of claim 1, wherein the GRAS gelator is a sorbitan alkanoate selected from the group consisting of sorbitan monostearate, sorbitan decanoate, sorbitan laurate, sorbitan caprylate, sorbitan myristate, sorbitan oleate, and combinations thereof.

12. The gel composition of claim 1, wherein the therapeutic, prophylactic, or diagnostic agent comprises a protein or a peptide, nucleic acid molecule, lipoprotein, lipid, or small molecule.

13. The gel composition of claim 1, comprising two or more agents, wherein at least one agent potentiates efficacy of the other agent.

14. The gel composition of claim 1, wherein solvent or unencapsulated agent is removed.

15. The gel composition of claim 14 wherein the solvent is removed by lyophilization or drying.

16. The gel composition of claim 1 wherein the gel composition is dispersed or broken up into pieces.

17. The gel composition of claim 1 in a sterile dosage unit kit.

18. The gel composition of claim 1, wherein the dosage unit comprises one or more containers for dry components and one or more containers for liquid components, which are mixed together to form the self-assembled gel composition.

19. The gel composition of claim 1 comprising a pharmaceutically acceptable carrier, optionally wherein the gel composition or the purified gel composition is homogenized or otherwise dispersed in the pharmaceutically acceptable carrier.

20. The gel composition of claim 1 wherein the gel composition is in the form of dispersed particles, sheets or tapes formed by breaking or dispersing the gel.

21. The gel composition of claim 16 wherein the carrier is a bandage, wound dressing, or patch.

22. A method of forming the gel composition of claim 1, comprising:
forming a homogenous solution comprising a GRAS gelator having a molecular weight of 2,500 or less and a therapeutic, prophylactic, or diagnostic agent in a medium comprising water or an aqueous solution and an organic solvent, in the absence of heating to above 37° C.

23. The method of claim 22, comprising formed the gel by heating the homogeneous solution to 37° C. or 25° C., then cooling.

24. The method of claim 22, wherein the GRAS gelator is present in a concentration of at least 4 wt/vol % or greater in the gelation medium, and the organic solvent is between 15% and 50% in volume of the gelation medium.

25. The method of claim 22, wherein solvent or unencapsulated agent is removed by lyophilization or drying.

26. The method of claim 22 wherein the gel composition is dispersed or broken up into pieces.

27. The method of claim 22 wherein the gel composition is packaged into a sterile dosage unit kit for administration topically or by injection.

28. The method of claim 27, comprising mixing the dry components and liquid components, for administration at a site in need thereof.

29. A method of administering therapeutic, prophylactic or diagnostic agent comprising administering to an individual in need thereof the gel composition of claim 1.

30. The method of claim 29 wherein the gel is administered by injection or implantation.

31. The method of claim 29 wherein the gel is administered topically.

32. The method of claim 31 wherein the gel is administered as a powder or dry dispersion.

33. The method of claim 31 wherein the gel, optionally as a powder, is administered to a mucosal surface selected from the group consisting of nasal mucosal, oral mucosal, buccal mucosal, pulmonary mucosa, vaginal mucosal, intestinal mucosa, and rectal mucosa.

34. The method of claim 31 wherein the gel, optionally dried or as a powder or particulate formulation, is incorporated into or onto a wound covering or dressing and applied to a wound.
